(19) United States
(12) Patent Application Publication
(10) Pub. No.: US 2021/0085359 A1
(43) Pub. Date: Mar. 25, 2021

(54) ARTICULATING CURETTE

(71) Applicant: Spineology Inc., St. Paul, MN (US)

(72) Inventor: Joseph GLEASON, Eagan, MN (US)

(21) Appl. No.: 17/027,619

(22) Filed: Sep. 21, 2020

Related U.S. Application Data
(60) Provisional application No. 62/903,480, filed on Sep. 20, 2019.

(51) Int. Cl. A61B 17/3207; A61B 17/16

(52) U.S. Cl. CPC A61B 17/320708; A61B 17/1604

(57) ABSTRACT

A shaper for reaming tissue having a single articulating cutting head can be operably connected to a main shaft. The cutting head can be inverted by rotating the main shaft 180 degrees so that the remaining half-circle of tissue can be removed for a complete discectomy or other surgical procedure. The main shaft can be rotationally locked so that rotation does not occur until the user actuates a release mechanism.

### PRIORITY

This application claims the benefit of U.S. Provisional Application Ser. No. 62/903,480, filed on Sep. 20, 2019, which is hereby incorporated herein by reference in its entirety.

### FIELD

The invention relates to an apparatus and method for removing, reaming, debriding and/or resecting tissue and/or bone. In particular, the present invention is directed to an articulating curette or shaper that may be incrementally deployed to create a variety of cavity shapes. The apparatus and method of the present invention may be especially useful in medical procedures such as orthopedic surgery.

### BACKGROUND

A variety of tools are available for surgeons to remove spinal disc tissue and/or bony tissue during surgery in the spinal region.

Pituitary rongeurs and curettes are frequently used instruments to remove tissue. Some examples of these instruments are described in the following U.S. patents: U.S. Pat. No. 6,200,320 to Michelson; U.S. Pat. No. 6,142,997 to Michelson; U.S. Pat. No. 5,961,531 to Weber et al.; U.S. Pat. No. 5,766,177 to Lucas-Dean et al.; U.S. Pat. No. 5,653,713 to Michelson; U.S. Pat. No. 5,484,441 to Koros et al.; U.S. Pat. No. 5,451,227 to Michelson; U.S. Pat. No. 5,312,407 to Carter; U.S. Pat. No. 5,026,375 to Linovitz et al. U.S. Pat. No. 5,061,269 to Muller; U.S. Pat. No. 4,990,148 to Worrick, III et al.; U.S. Pat. No. 4,777,948 to Wright; U.S. Pat. No. 4,733,663 to Farely; U.S. Pat. No. 4,722,338 to Wright et al.; U.S. Pat. No. 3,902,498 to Niederer; U.S. Pat. No. 3,628,524 to Jamshidi and U.S. Pat. No. 2,984,241 to Carlson.

The use of rongeurs and curettes tends to leave behind fragments of tissue. Further, because these rongeurs and curettes require multiple passes, the operation may be prolonged, possibly leading to increased bleeding and higher infection rates. Many pituitary rongeurs utilize a single cutting blade at the end of a single, unopposed beam. Actuation of the beam, by means of a drive rod, tends to force the distal shaft to move away from the tissue being cut. An open section in the middle of the beam helps reduce this movement, but does not effectively eliminate the unwanted movement.

U.S. Pat. No. 5,445,639 to Kuslich et al., describes an intervertebral reamer used to ream out the interior of a degenerated disc to clean the interbody space. U.S. Pat. No. 6,383,188 to Kuslich et al., discloses an expandable reamer including a pair of opposing blades which have an expanded state and a retracted state. The blades are pivotally positioned at the distal end of a shaft assembly.

U.S. Pat. No. 6,575,978 to Peterson et al., discloses a circumferential resecting reamer tool. The reamer disclosed in the '978 patent is a multibladed cutting tool that circumferentially reams tissue. The cutting blades sweep through an arc creating a transverse cavity. U.S. Pat. No. 6,575,978 is hereby incorporated herein by reference in its entirety.

U.S. Pat. No. 5,928,239 to Mirza discloses a reamer which has a shaft and a cutting tip attached through a free rotating hinge such that high speed rotation allows the tip to be deflected outwardly to form a cavity. U.S. Pat. No. 5,591,170 to Spievack et al discloses a powered bone saw which inserts its cutting blade through a bored intramedullary canal.

U.S. Pat. Nos. 6,440,138 and 6,863,672 to Reiley et al., describe tools for creating cavities in bone wherein the tool includes various cutting tips carried on a shaft. The cutting tips disclosed include a rotatable loop, brush, or blade, a linear cutting blade and an energy transmitter. U.S. Pat. No. 6,923,813 to Phillips et al., discloses tools for creating voids in interior body regions. The tools include several different cutting tips which provide for rotational and translational cutting.

While the foregoing surgical tools are effective in creating cavities within a patient's body, it would be desirable to provide a tool for removal of tissue that is capable of more refined control, particularly of cavity shape, yet is simple to operate.

### SUMMARY

The present invention, according to certain embodiments, comprises a shaper for reaming tissue having a single articulating cutting head pivotally connected to a main shaft. According to one aspect, the cutting head can be inverted by rotating the main shaft 180 degrees so that the remaining half-circle of tissue can be removed for a complete discectomy. The main shaft can be rotationally locked so that rotation does not occur until the user actuates a release mechanism.

According to an example embodiment, an articulating curette can include a main shaft, a cutting tip pivotally disposed at a distal end of the main shaft, and a handle assembly. The handle assembly can be coupled to a proximal end of the main shaft such that the main shaft can rotate axially with respect to the handle assembly. The handle assembly can comprise a shaft rotating handle coupled to the main shaft. A pivot axis of the cutting tip can be normal to a rotational axis of the main shaft.

The handle assembly can further comprise a first notch sized and shaped to engage the shaft rotating handle in a first position such that the shaft rotating handle cannot rotate the main shaft when the shaft rotating handle is engaged with the first notch.

The shaft rotating handle can comprise an end stop at a proximal end of the shaft rotating handle and a stem disposed between the proximal end and a distal end of the shaft rotating handle, wherein a distal end of the shaft rotating handle is rotationally coupled to the main shaft, and wherein the stem is biased away from the end stop by a biasing member so that the stem engages the botch until a force is applied to the stem in a proximal direction sufficient to overcome the biasing member and thereby release the stem from the notch. The biasing member can be a spring or a compressible piece of material, such as a rubber washer.

The handle assembly can further comprise a second notch sized and shaped to engage the shaft rotating handle in a second position such that the shaft rotating handle cannot rotate the main shaft when the shaft rotating handle is engaged with the second notch. The first positon and the second position can be 180 degrees opposite each other. There may be no third notch to define a third position between the first and second positions. Or there may be a third or further notches to define additional positions between the first and second positions.

The handle assembly can comprise a channel spanning between the first positon and the second position, and wherein the channel terminates at a first end in the first notch and at a second end in the second notch.

The cutting tip can be coupled to the main shaft so that the cutting tip rotates along with the main shaft. The cutting tip can comprise a first cutting edge and a second cutting edge that is 180 degrees opposite to the first cutting edge.

According to an example embodiment, a method of removing tissues from a patient can include inserting a distal end of an articulating curette into a target location in the patient, pivoting a cutting head of the articulating curette in a first rotational direction in a first plane to remove tissue from the patient, and rotating a main shaft of the articulating curette with respect to a handle assembly of the articulating curette so that the cutting head will rotate in a second cutting plane.

The second cutting plane can be the same plane as the first cutting plane except that the cutting head pivots in a second rotational direction that is opposite the first rotational direction.

In a further example embodiment, a method of removing tissues from a patient can include inserting a distal end of an articulating curette into a target location in the patient, pivoting a cutting head of the articulating curette in a first rotational direction to remove tissue from the patient, and rotating a main shaft of the articulating curette with respect to a handle assembly of the articulating curette so that the cutting head will be inverted and rotate in a second rotational direction opposite the first rotational direction to remove tissue from the patient.

The step of rotating the main shaft can include rotating a rotation actuator handle about a rotational axis of the main shaft.

The rotation actuator handle can be prevented from rotating about the rotational axis of the main shaft until after the rotation actuator handle has been released from a notch that is engaged by the rotation actuator handle. The rotation actuator handle can be released from the notch by pulling proximally on a stem of the rotation actuator handle towards an end stop. The stem away from the end stop.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 is a perspective view of a shaper in accordance with embodiments of the present invention.

FIG. 2 is another perspective view of the shaper of FIG. 1 in accordance with embodiments of the present invention.

FIG. 3 is a side view of a distal end portion of a shaper in accordance with embodiments of the present invention.

FIG. 4 is another side view of a distal end portion of a shaper in accordance with embodiments of the present invention.

FIG. 5 is a side view of a distal end portion of a shaper in accordance with embodiments of the present invention.

FIG. 6 is a side view of a distal end portion of a shaper in accordance with embodiments of the present invention.

FIG. 7 is a side view of a distal end portion of a shaper in accordance with embodiments of the present invention.

FIG. 8 is a side view of a distal end portion of a shaper in accordance with embodiments of the present invention.

FIG. 9 is a side view of a distal end portion of a shaper in accordance with embodiments of the present invention.

FIG. 10 is a side view of a distal end portion of a shaper in accordance with embodiments of the present invention.

FIG. 11 is a side view of a distal end portion of a shaper in accordance with embodiments of the present invention.

FIG. 12 is a side view of a distal end portion of a shaper in accordance with embodiments of the present invention.

FIG. 13 is a side view of a distal end portion of a shaper in accordance with embodiments of the present invention.

FIG. 14 is a side view of a distal end portion of a shaper in accordance with embodiments of the present invention.

FIG. 15 is a side view of a distal end portion of a shaper in accordance with embodiments of the present invention.

FIG. 16 is a side view of a distal end portion of a shaper in accordance with embodiments of the present invention.

FIG. 17 is a side view of a distal end portion of a shaper in accordance with embodiments of the present invention.

FIG. 18 is a side view of a proximal end portion of a shaper in accordance with embodiments of the present invention.

FIG. 19 is a side view of a proximal end portion of a shaper in accordance with embodiments of the present invention.

FIG. 20 is a side view of a distal end portion of a shaper in accordance with embodiments of the present invention.

FIG. 21 is a side view of a distal end portion of a shaper in accordance with embodiments of the present invention.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the spirit and scope of the invention as defined by the appended claims. For illustrative purposes, cross-hatching, dashing or shading in the figures is provided to demonstrate sealed portions and/or integrated regions or devices for the package.

### DETAILED DESCRIPTION

In the following descriptions, the present invention will be explained with reference to example embodiments thereof. However, these example embodiments are not intended to limit the present invention to any specific example, embodiment, environment, applications or particular implementations described in these embodiments. Therefore, description of these embodiments is only for purpose of illustration rather than to limit the present invention.

It should be appreciated that, in the following embodiments and the attached drawings, elements unrelated to the present invention are omitted from depiction; and dimensional relationships among individual elements in the attached drawings are illustrated only for ease of understanding, but not to limit the actual scale.

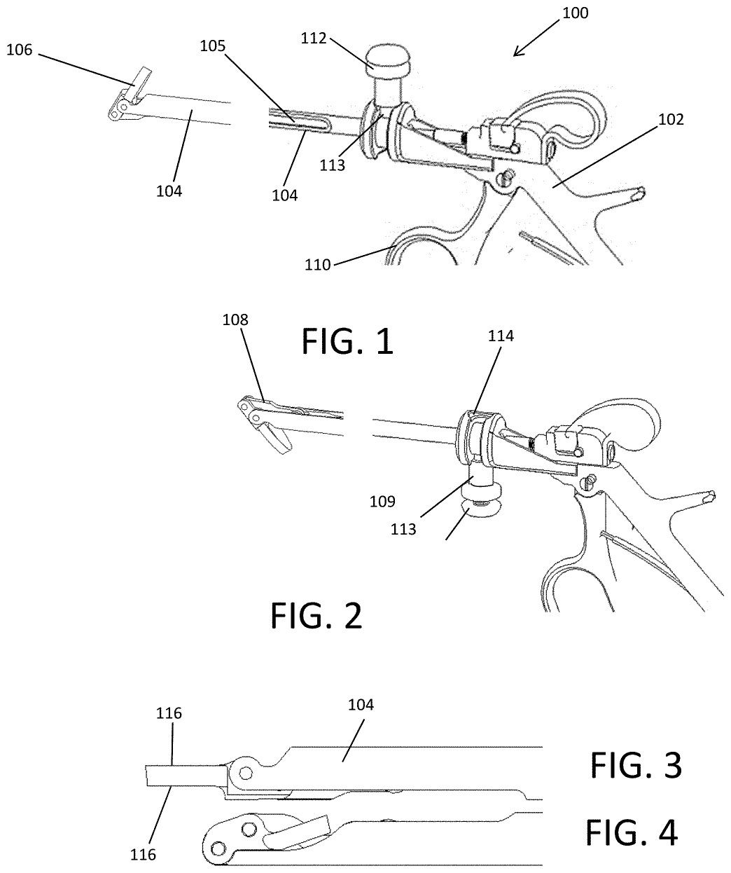

Referring to FIGS. 1-2, the articulating curette 100 (also referred to as a shaper) generally comprises a handle assembly 102 disposed on a proximal end of a hollow main shaft 104 and a cutting tip 106 pivotally disposed at the distal end of the main shaft 104. A channel 105 can be formed longitudinally through the main shaft 104.

A distal end of an actuator shaft 108 is coupled to an end of the cutting tip 106 opposite the cutting edge thereof. The proximal end of the actuator shaft 108 is coupled to a cutting actuator handle 110 of the handle assembly 102. The actuator shaft 108 extends longitudinally through the main shaft 104.

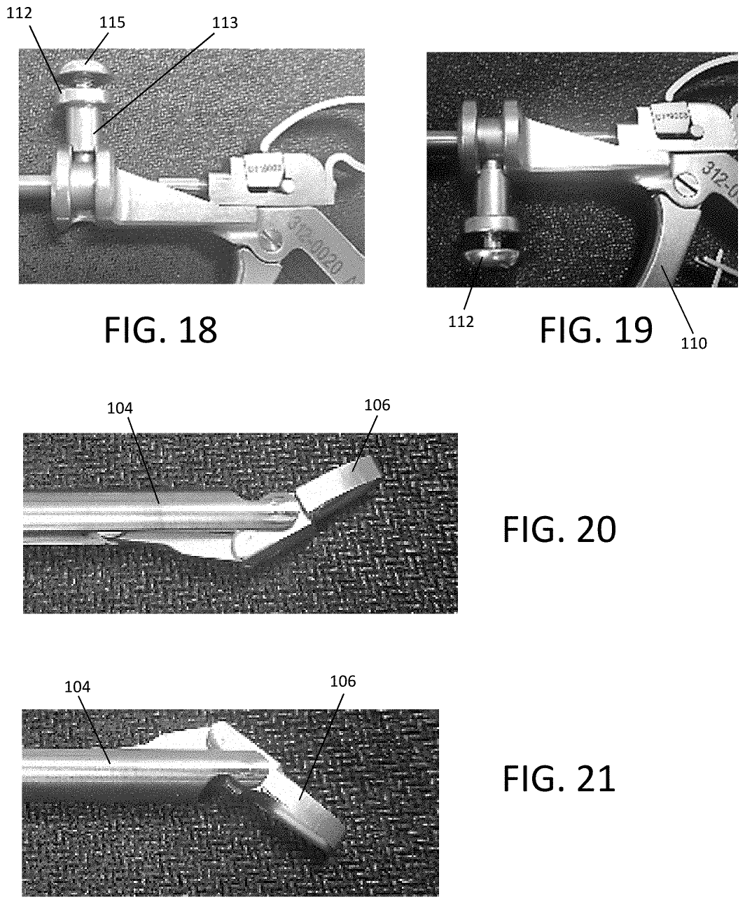

The main shaft 104 is rotationally coupled to the handle assembly so that the main shaft 104 can rotate 180 degrees about its longitudinal axis. A rotation actuator handle 112 is disposed in a curved slot 109 located adjacent to a distal end of the handle assembly 102 and is coupled to the main shaft 104. Thus, the handle 112 can be used by the operator to selectably rotate the main shaft 104 up to 180 degrees from a normal (or first) orientation (FIG. 1) to an inverted (or second) orientation (FIG. 2) that is 180 degrees opposite that of the normal orientation. FIGS. 18-19 show the handle 112 in the normal position and the inverted positon, respectively.

The stem 113 or distal end of the rotation actuator handle 112 (opposite the proximal or bulbous end that the user can grasp) mates with a correspondingly-shaped recess 114 defined in the curved slot 109 slot through which the rotation actuator handle 112 moves. This ensures that the shaft 104 does not unintentionally rotate. A similar recess is also defined at the fully-inverted position so that the rotation actuator handle 112 is secured in the inverted position.

The stem 113 can comprise a spring loaded lockout mechanism. This feature requires the user to pull axially on the stem 113 of the handle 112 in the proximal direction towards an end stop 115 to disengage the stem 113 from the recess 114 before the handle 112 can be rotated. This locking mechanism configuration further prevents the main shaft 104 from unintentionally rotating.

The slot 109 can be configured with additional recesses 114 to provide for more than the depicted two recesses arrayed 180 degrees opposite one another. For example, a third slot can be placed at 90 degrees offset from the first rotational position. The slot 109 can sized to allow for more than 180 degrees of rotation of the main shaft 104 (e.g. 270 or 360 degrees of rotation). The slot 109 can also be sized to allow for less than 180 degrees of rotation of the main shaft 104 (e.g. 90 degrees of rotation).

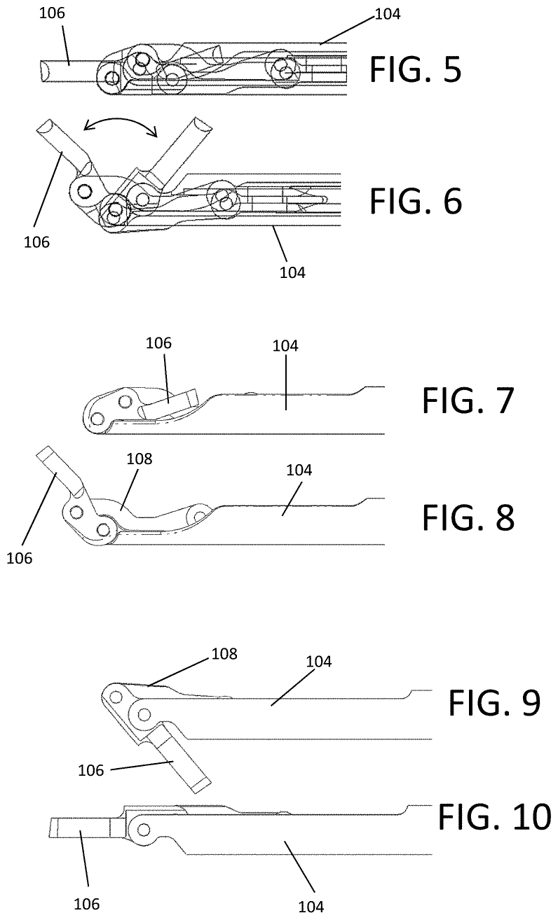

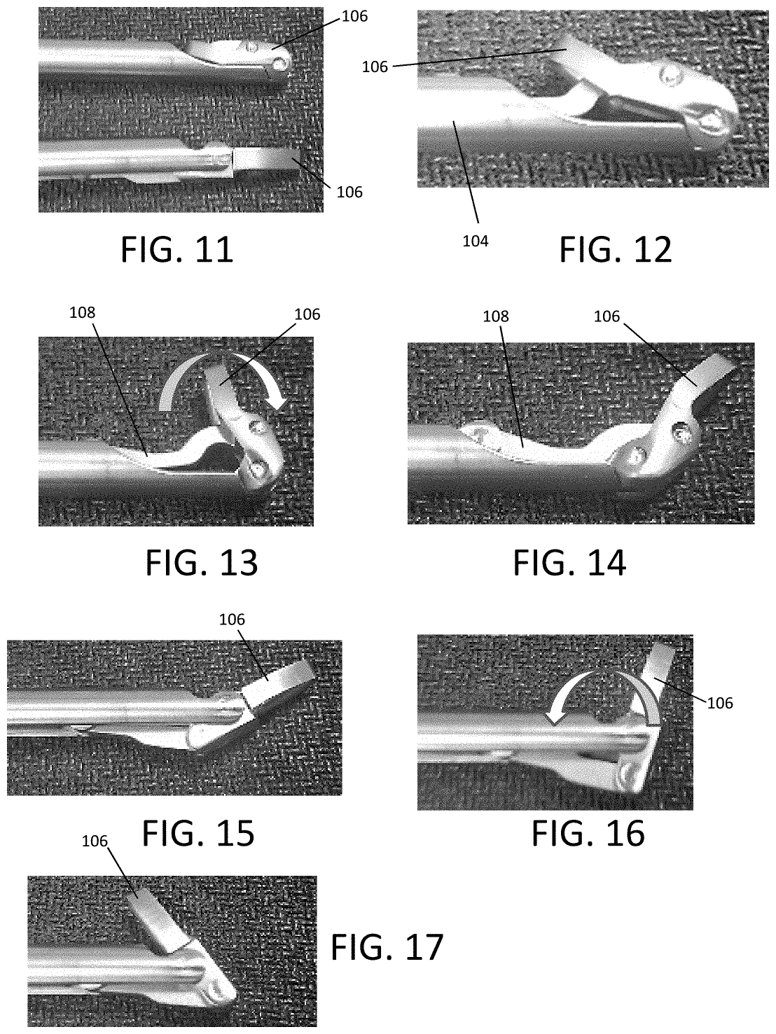

Referring further to FIGS. 3-4, 5-6, 7-8 and 9-10, 11-17 and 20-21, the cutting tip 106 has a cutting edge 116 defined on one or both of the top and bottom sides thereof. The two-sided configuration (shown throughout the figures) allows the cutting tip 106 to cut using both the forward and reverse rotational directions. Note that FIGS. 9-10 and 21 show the cutting tip with the main shaft 104 rotated into the inverted position.

In use, the user inserts the distal end of the shaper 100 into the target location in the patient's tissues such that the cutting tip 106 is in the desired location to remove tissue. Then the cutting actuator handle 118 is actuated to pivot and move the cutting tip 106 forward to create a first half-circle portion of a cavity in the patient's tissues. Next, the user pulls proximally on the handle 112 in an axial direction to release the rotation actuator handle 112 from its recess 114. Then the user rotates the rotation actuator handle 112 to the desired position (e.g., the inverted position) so that the cutting tip 106 pivots in the reverse direction to remove the second half-circle portion of a cavity in the patient's tissues. The result is that a complete or substantially complete circular or arcuate cavity is formed such as is desired in a discectomy procedure.

The spring or resilient member inside of the stem 113 of the rotation actuator handle 112 automatically pulls the handle 112 axially downward in its distal direction into the respective recess 114 and maintains the handle 112 there until the user pulls the handle 112 with sufficient force to initiate a deliberate rotational movement of the main shaft 104.

The two-sided cutting tip 106 and ability to rotate the tip 106 from a normal to an inverted position allows the user to easily invert the cutting tip, without rotating the actuator handle 110 of the handle assembly 102 itself, by rotating the main shaft 180 degrees so that the remaining half-circle of tissue can be removed for a complete discectomy.

The articulating curette 100 can also be adapted to other surgical procedures where a sweeping cutting motion is desired.

The articulating curette can be packaged as part of a kit and system that includes an implant. The kit and system embodiments can also include additional surgical instruments.

US Patent Application Pub. No. 2009/0054898 A1 is hereby incorporated by reference in its entirety herein.

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached hereto.

What is claimed is:

1. An articulating curette, comprising:
a main shaft;
a cutting tip pivotally disposed at a distal end of the main shaft; and
a handle assembly coupled to a proximal end of the main shaft such that the main shaft can rotate axially with respect to the handle assembly, the handle assembly comprising a shaft rotating handle coupled to the main shaft,
wherein a pivot axis of the cutting tip is normal to a rotational axis of the main shaft.

2. The articulating curette of claim 1, wherein the handle assembly further comprises a first notch sized and shaped to engage the shaft rotating handle in a first position such that the shaft rotating handle cannot rotate the main shaft when the shaft rotating handle is engaged with the first notch.

3. The articulating curette of claim 2, wherein the shaft rotating handle comprises an end stop at a proximal end of the shaft rotating handle and a stem disposed between the proximal end and a distal end of the shaft rotating handle, wherein a distal end of the shaft rotating handle is rotationally coupled to the main shaft, and wherein the stem is biased away from the end stop by a biasing member so that the stem engages the botch until a force is applied to the stem in a proximal direction sufficient to overcome the biasing member and thereby release the stem from the notch.

4. The articulating curette of claim 3, wherein the biasing member is a spring.

5. The articulating curette of claim 2, wherein the handle assembly further comprises a second notch sized and shaped to engage the shaft rotating handle in a second position such that the shaft rotating handle cannot rotate the main shaft when the shaft rotating handle is engaged with the second notch.

6. The articulating curette of claim 5, wherein the first positon and the second position are 180 degrees opposite each other.

7. The articulating curette of claim 5, wherein the first positon and the second position are 180 degrees opposite each other, and wherein there is no third notch to define a third position.

8. The articulating curette of claim 5, wherein the handle assembly comprises a channel spanning between the first positon and the second position, and wherein the channel terminates at a first end in the first notch and at a second end in the second notch.

9. The articulating curette of claim 1, wherein the main shaft can rotate between a first positon and second position that is 180 degrees opposite the first position, wherein the cutting tip is coupled to the main shaft so that the cutting tip rotates along with the main shaft.

10. The articulating curette of claim 1, wherein the cutting tip comprises a first cutting edge and a second cutting edge that is 180 degrees opposite to the first cutting edge.

11. A method of removing tissues from a patient, the method comprising:
inserting a distal end of an articulating curette into a target location in the patient;
pivoting a cutting head of the articulating curette in a first rotational direction in a first plane to remove tissue from the patient; and
rotating a main shaft of the articulating curette with respect to a handle assembly of the articulating curette so that the cutting head will rotate in a second cutting plane.

12. The method of claim 11, wherein the second cutting plane is the same plane as the first cutting plane except that the cutting head pivots in a second rotational direction that is opposite the first rotational direction.

13. The method of claim 11, wherein the step of rotating the main shaft includes rotating a rotation actuator handle about a rotational axis of the main shaft.

14. The method of claim 13, further comprising preventing the rotation actuator handle from rotating about the rotational axis of the main shaft until after the rotation actuator handle has been released from a notch that is engaged by the rotation actuator handle.

15. The method of claim 14, further comprising releasing the rotation actuator handle from the notch by pulling proximally on a stem of the rotation actuator handle towards an end stop.

16. The method of claim 15, further comprising biasing the stem away from the end stop.

17. A method of removing tissues from a patient, the method comprising:
inserting a distal end of an articulating curette into a target location in the patient;
pivoting a cutting head of the articulating curette in a first rotational direction to remove tissue from the patient; and
rotating a main shaft of the articulating curette with respect to a handle assembly of the articulating curette so that the cutting head will be inverted and rotate in a second rotational direction opposite the first rotational direction to remove tissue from the patient.

18. The method of claim 17, wherein the step of rotating the main shaft includes rotating a rotation actuator handle about a rotational axis of the main shaft, and the method further comprises preventing the rotation actuator handle from rotating about the rotational axis of the main shaft until after the rotation actuator handle has been released from a notch that is engaged by the rotation actuator handle.

19. The method of claim 18, further comprising releasing the rotation actuator handle from the notch by pulling proximally on a stem of the rotation actuator handle towards an end stop.

20. The method of claim 19, further comprising biasing the stem away from the end stop.
